(19) United States
(12) Patent Application Publication
(10) Pub. No.: US 2006/0058823 A1
(43) Pub. Date: Mar. 16, 2006

(54) Handpiece pumping chamber

(76) Inventors: Ramon C. Dimalanta, Rancho Santa Margarita, CA (US); Ziad R. Ghannoum, Trabuco Canyon, CA (US); Amir H. Khashayar, Laguna Nigel, CA (US); Sean C. Madden, Mission Viejo, CA (US); Glenn Sussman, Laguna Nigel, CA (US); John R. Underwood, Laguna Nigel, CA (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/940,400

(22) Filed: Sep. 14, 2004

(51) Int. Cl. A61B 17/32

(52) U.S. Cl. 606/167

(57) ABSTRACT

A surgical handpiece having at least two lumens or tubes mounted to a body. At least one tube is used for aspiration and at least one other tube is used to inject heated surgical fluid for performing a surgical procedure, such as an ophthalmic or otic surgical procedure. A portion of the second tube may form a pumping chamber. The pumping chamber works by boiling a small volume of the surgical fluid. As the fluid boils, it expands rapidly, thereby propelling the liquid downstream of the pumping chamber out of the second tube. The pumping chamber may use a heating filament or a laser to heat the surgical fluid within the boiling chamber, thereby creating a pressure wave that travels down the second tube.

### BACKGROUND OF THE INVENTION

This invention relates generally to the field of ophthalmic and otic surgery and more particularly to a pumping chamber for a handpiece for ophthalmic and otic surgery.

The human eye in its simplest terms functions to provide vision by transmitting light through a clear outer portion called the cornea, and focusing the image by way of the lens onto the retina. The quality of the focused image depends on many factors including the size and shape of the eye, and the transparency of the cornea and lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. During this procedure, a thin phacoemulsification cutting tip is inserted into the diseased lens and vibrated ultrasonically. The vibrating cutting tip liquifies or emulsifies the lens so that the lens may be aspirated out of the eye. The diseased lens, once removed, is replaced by an artificial lens.

A typical ultrasonic surgical device suitable for ophthalmic procedures consists of an ultrasonically driven handpiece, an attached cutting tip, and irrigating sleeve and an electronic control console. The handpiece assembly is attached to the control console by an electric cable and flexible tubings. Through the electric cable, the console varies the power level transmitted by the handpiece to the attached cutting tip and the flexible tubings supply irrigation fluid to and draw aspiration fluid from the eye through the handpiece assembly.

The operative part of the handpiece is a centrally located, hollow resonating bar or horn directly attached to a set of piezoelectric crystals. The crystals supply the required ultrasonic vibration needed to drive both the horn and the attached cutting tip during phacoemulsification and are controlled by the console. The crystal/horn assembly is suspended within the hollow body or shell of the handpiece by flexible mountings. The handpiece body terminates in a reduced diameter portion or nosecone at the body's distal end. The nosecone is externally threaded to accept the irrigation sleeve. Likewise, the horn bore is internally threaded at its distal end to receive the external threads of the cutting tip. The irrigation sleeve also has an internally threaded bore that is screwed onto the external threads of the nosecone. The cutting tip is adjusted so that the tip projects only a predetermined amount past the open end of the irrigating sleeve.

In use, the ends of the cutting tip and irrigating sleeve are inserted into a small incision of predetermined width in the cornea, sclera, or other location. The cutting tip is ultrasonically vibrated along its longitudinal axis within the irrigating sleeve by the crystal-driven ultrasonic horn, thereby emulsifying the selected tissue in situ. The hollow bore of the cutting tip communicates with the bore in the horn that in turn communicates with the aspiration line from the handpiece to the console. A reduced pressure or vacuum source in the console draws or aspirates the emulsified tissue from the eye through the open end of the cutting tip, the cutting tip and horn bores and the aspiration line and into a collection device. The aspiration of emulsified tissue is aided by a saline flushing solution or irrigant that is injected into the surgical site through the small annular gap between the inside surface of the irrigating sleeve and the cutting tip.

Recently, a new cataract removal technique has been developed that involves the injection of hot (approximately 45° C. to 105° C.) water or saline to liquefy or gellate the hard lens nucleus, thereby making it possible to aspirate the liquefied lens from the eye. Aspiration is conducted with the injection of the heated solution and the injection of a relatively cool solution, thereby quickly cooling and removing the heated solution. This technique is more fully described in U.S. Pat. No. 5,616,120 (Andrew, et al.), the entire contents of which is incorporated herein by reference. The apparatus disclosed in the publication, however, heats the solution separately from the surgical handpiece. Temperature control of the heated solution can be difficult because the fluid tubings feeding the handpiece typically are up to two meters long, and the heated solution can cool considerably as it travels down the length of the tubing.

Several prior art devices solve this problem by heating the fluid in the handpiece. For example, the device disclosed in U.S. Pat. No. 6,398,759 B1 (Sussman, et al.), which is commercially available as the AQUALASE® handpiece from Alcon Laboratories, Inc., Fort Worth, Tex., uses a pair of spaced electrodes. The conductive nature of the surgical fluid allows electricity to pass between the electrodes, thereby boiling the surgical fluid. The device disclosed in U.S. Pat. No. 5,885,243 (Capetan, et al.), which uses an insulated heating element and the device disclosed in U.S. Pat. No. 5,865,790 (Bair), that passes the surgical fluid through a porous medium at high pressure in order to heat the fluid, cannot produce high frequency pressure pulses. Still another device is disclosed in U.S. Pat. No. 6,440,103 (Hood, et al.) which uses a heating element located at the distal tip of the handpiece. Locating the active heating element at the distal end of the handpiece tip causes the heating element to enter the eye during surgery, raising possible safety issues.

Therefore, a need continues to exist for a control system for a surgical handpiece that can heat internally the solution used to perform surgical procedures to produce high frequency pressure pulses.

### BRIEF SUMMARY OF THE INVENTION

The present invention improves upon the prior art by providing a surgical handpiece having at least two lumens or tubes mounted to a body. At least one tube is used for aspiration and at least one other tube is used to inject heated surgical fluid for performing a surgical procedure, such as an ophthalmic or otic surgical procedure. A portion of the second tube may form a pumping chamber. The pumping chamber works by boiling a small volume of the surgical fluid. As the fluid boils, it expands rapidly, thereby propelling the liquid downstream of the pumping chamber out of the second tube. The pumping chamber may use a heating filament or a laser to heat the surgical fluid within the boiling chamber, thereby creating a pressure wave that travels down the second tube.

Accordingly, one objective of the present invention is to provide a surgical handpiece having at least two tubes.

Another objective of the present invention is to provide a surgical handpiece having a pumping chamber with heating filament.

Another objective of the present invention is to provide a surgical handpiece having a pumping chamber with a laser.

Another objective of the present invention is to provide a surgical handpiece having a device for delivering the surgical fluid through the handpiece in pulses.

These and other advantages and objectives of the present invention will become apparent from the detailed description and claims that follow.

### BRIEF DESCRIPTION OF THE DRAWINGS

FIG. 1 is a partial cross-sectional view of a first embodiment of the handpiece of the present invention.

FIG. 2 is an exploded partial cross-section taken at circle 2 in FIG. 1.

FIG. 3 is a partial cross-sectional view of a second embodiment of the handpiece of the present invention.

FIG. 4 is an exploded partial cross-section taken at circle 4 in FIG. 3.

### DETAILED DESCRIPTION OF THE INVENTION

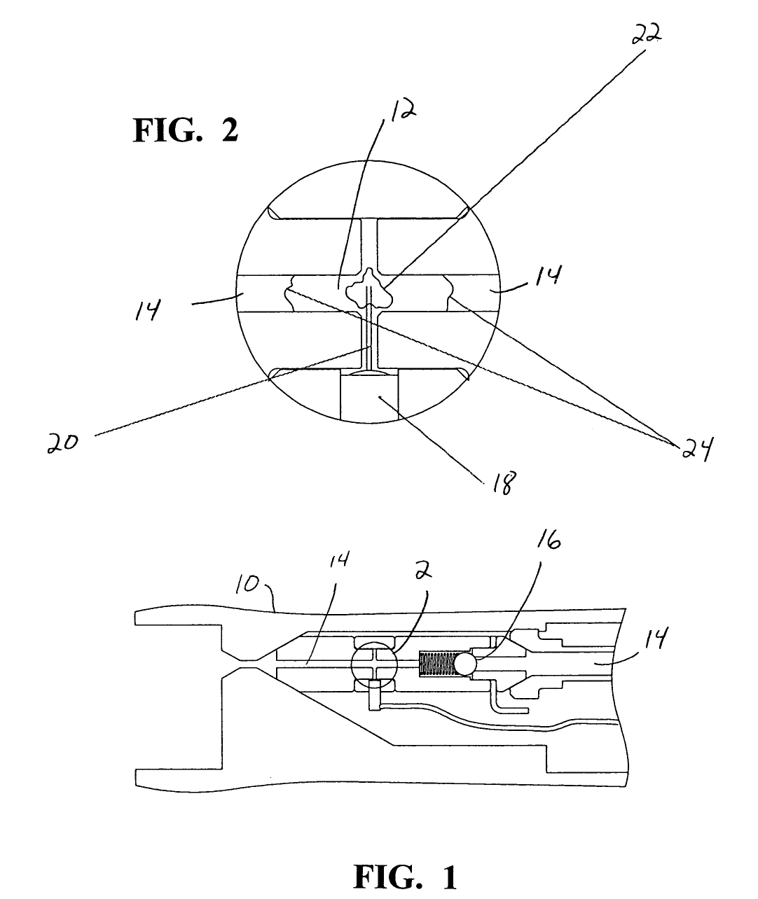

With reference to FIGS. 1 and 2, handpiece 10 of the present invention is similar in construction to well-known handpieces, such as the AQUALASE® handpiece from Alcon Laboratories, Inc., Fort Worth, Tex., and may be made from plastic, titanium or stainless steel. Handpiece 10 contains pumping chamber 12, which forms part of tube 14. Surgical irrigation fluid enters pumping chamber 12 under pressure through check valve 16. Laser 18 is arranged so that focused beam 20 of laser radiation enters pumping chamber 12, boiling the surgical fluid and forming vapor bubble 22. Rapid expansion of vapor bubble 22 produces two pressure wavefronts 24, which are prevented from moving proximally down tube 14 by check valve 16. As a result, the pressure wavefronts 24 move distally down tube 14 and out any suitable tip mounted on handpiece 10, such tips being known in the art and commercially available from Alcon Laboratories, Inc., Fort Worth, Tex. Subsequent pulses of laser 18 form sequential vapor bubbles 22 that move surgical fluid down tube 14. The size and pressure of the fluid pulse obtained by pumping chamber 12 can be varied by varying the length, timing and/or power of laser 18 and by varying the dimensions of pumping chamber 12. In addition, the surgical fluid may be preheated prior to entering pumping chamber 12. Preheating the surgical fluid will decrease the power required by pumping chamber 12 and/or increase the speed at which pressure pulses can be generated.

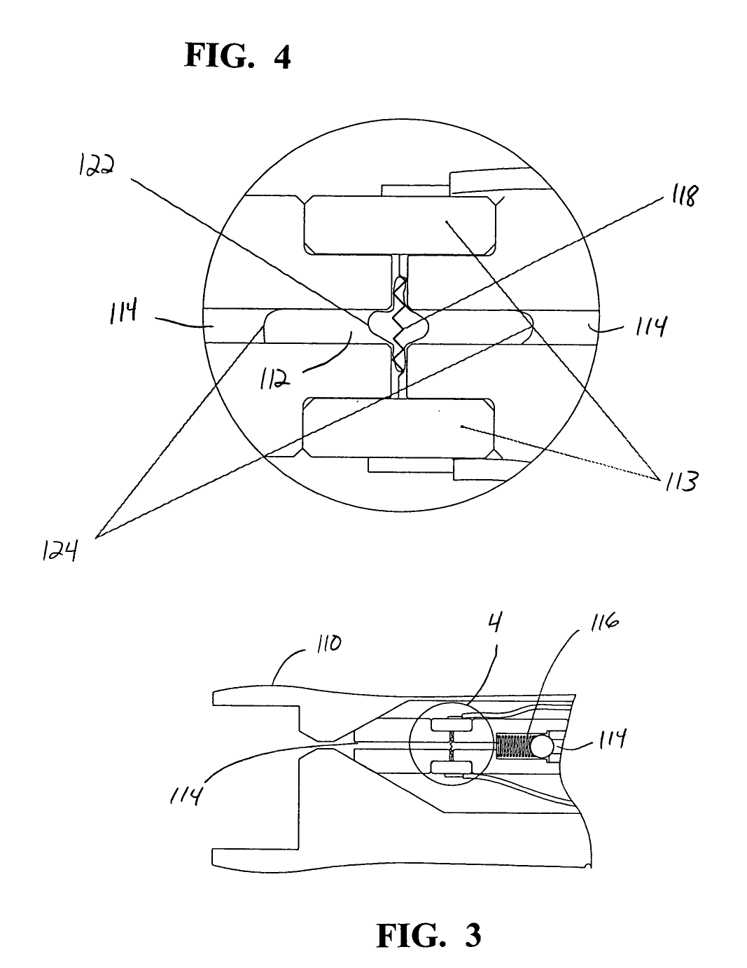

With reference to FIGS. 3 and 4, handpiece 110 of the present invention is similar in construction to well-known handpieces, such as the AQUALASE® handpiece from Alcon Laboratories, Inc., Fort Worth, Tex., and may be made from plastic, titanium or stainless steel. Handpiece 110 contains pumping chamber 112, which forms part of tube 114. Surgical irrigation fluid enters pumping chamber 112 under pressure through check valve 116. Heating filament 118 is arranged within pumping chamber 112 between electrodes 113 so that filament 118 boils the surgical fluid within pumping chamber 112, forming vapor bubble 122. Rapid expansion of vapor bubble 122 produces two pressure wavefronts 124, which are prevented from moving proximally down tube 114 by check valve 116. As a result, the pressure wavefronts 124 move distally down tube 114 and out any suitable tip mounted on handpiece 110, such tips being known in the art and commercially available from Alcon Laboratories, Inc., Fort Worth, Tex. Subsequent pulses of electrodes 113 into filament 118 form sequential vapor bubbles 122 that move surgical fluid down tube 114. The size and pressure of the fluid pulse obtained by pumping chamber 112 can be varied by varying the length, timing and/or power of electrodes 113 and by varying the dimensions of pumping chamber 112. In addition, the surgical fluid may be preheated prior to entering pumping chamber 112. Preheating the surgical fluid will decrease the power required by pumping chamber 112 and/or increase the speed at which pressure pulses can be generated.

While multiple embodiments of the handpiece of the present invention are disclosed, any handpiece producing adequate pressure pulse force, rise time and frequency may also be used. For example, any suitable handpiece producing a pressure pulse force of between 0.03 grams and 50.0 grams (between 1 gram and 50.0 grams being preferred), with a rise time of between 1 gram/second and 50,000 grams/second (with between 500 grams/second and 50,000 grams/second being preferred) and a frequency of between 1 Hz and 200 Hz may be used, with between 10 Hz and 100 Hz being most preferred. The pressure pulse force and frequency may be varied with the hardness of the material being removed. For example, the inventors have found that a lower frequency with a higher pulse force is more efficient at debulking and removing the relatively hard nuclear material, with a higher frequency and lower pulse force being useful in removing softer epinuclear and cortical material. Infusion pressure, aspiration flow rate and vacuum limit are similar to current phacoemulsification techniques.

Any of a number of methods can be employed to order limit the amount of heat introduced into the eye. For example, the pulse train duty cycle of the heated solution can be varied so that the total amount of heated solution introduced into the eye does not vary with the pulse frequency. Alternatively, the aspiration flow rate can be varied as a function of pulse frequency so that as pulse frequency increases aspiration flow rate increases proportionally.

This description is given for purposes of illustration and explanation. It will be apparent to those skilled in the relevant art that changes and modifications may be made to the invention described above without departing from its scope or spirit. For example, it will be recognized by those skilled in the art that the present invention may be combined with ultrasonic and/or rotating cutting tips to enhance performance.

We claim:

1. A handpiece, comprising:
a) a pumping chamber; and
b) a laser emitting radiation, the laser being arranged relative to the pumping chamber so that the radiation emitted from the laser is focused within the pumping chamber.

2. A handpiece, comprising:
a) a pumping chamber; and
b) a heating filament connected to a pair of electrodes, the electrodes located adjacent to the pumping chamber and the filament located within the pumping chamber.
